(19) United States
(12) Patent Application Publication
(10) Pub. No.: US 2003/0045484 A1
(43) Pub. Date: Mar. 6, 2003

(54) Methods for preparing purified daptomycin

(76) Inventors: Dennis Keith, Montclair, NJ (US); Chandrika Govardhan, Lexington, MA (US); Nazer Khalaf, Worcester, MA (US)

Correspondence Address: FISH & RICHARDSON PC, 225 FRANKLIN ST, BOSTON, MA, 02110, US

(21) Appl. No.: 10/024,701

(22) Filed: Dec. 17, 2001

Related U.S. Application Data
(60) Provisional application No. 60/256,268, filed on Dec. 18, 2000.
(60) Provisional application No. 60/274,741, filed on Mar. 9, 2001.
(60) Provisional application No. 60/340,525, filed on Dec. 13, 2001.
(60) Provisional application No. 60/341,315, filed on Dec. 13, 2001.

(51) Int. Cl. C07H 17/08; A61K 31/7048

(52) U.S. Cl. 514/28; 536/7.1

(57) ABSTRACT

The present invention relates to methods of providing crystalline and crystal-like forms of daptomycin, a lipopeptide antibiotic with potent bactericidal activity against gram-positive bacteria, including strains that are resistant to conventional antibiotics. The present invention relates to methods of purifying daptomycin.

### CROSS-REFERENCE TO RELATED APPLICATIONS

The present application claims the benefit of United States Provisional Application No. 60/256,268, filed Dec. 18, 20000; Serial Number 60/274,741, filed Mar. 9, 2001; Serial Number ______ filed Dec. 13, 2001; and Serial Number ______ filed Dec. 13, 2001.

### TECHNICAL FIELD OF THE INVENTION

The present invention relates to methods of purifying daptomycin, an antibiotic with potent bactericidal activity against gram-positive bacteria, including strains that are resistant to conventional antibiotics. The present invention also relates to processes for preparing crystalline or crystal-like forms of daptomycin and to methods of purifying daptomycin.

### BACKGROUND OF THE INVENTION

The rapid increase in the incidence of gram-positive infections—including those caused by antibiotic-resistant bacteria—has sparked renewed interest in the development of novel classes of antibiotics. One such class is the lipopeptide antibiotics, which includes daptomycin. Daptomycin has potent bactericidal activity in vitro against clinically relevant gram-positive bacteria that cause serious and life-threatening diseases. These bacteria include, but are not limited to, resistant pathogens, such as vancomycin-resistant enterococci (VRE), methicillin-resistant Staphylococcus aureus (MRSA), glycopeptide intermediary susceptible Staphylococcus aureus (GISA), coagulase-negative staphylococci (CNS), and penicillin-resistant Streptococcus pneumoniae (PRSP), for which there are very few therapeutic alternatives. See, e.g., Tally et al., 1999, Exp. Opin. Invest. Drugs 8:1223-1238. Daptomycin's inhibitory effect is a rapid, concentration-dependent bactericidal effect in vitro and in vivo, and a relatively prolonged concentration-dependent post-antibiotic effect in vivo.

Daptomycin is described by Baltz in Biotechnology of Antibiotics 2nd Ed., ed. W. R. Strohl (New York: Marcel Dekker, Inc.), 1997, pp. 415-435. Daptomycin, also known as LY 146032, is a cyclic lipopeptide antibiotic that can be derived from the fermentation of Streptomyces roseosporus. Daptomycin is a member of the factor A-21978C₀ type antibiotics of S. roseosporus and is comprised of a decanoyl side chain linked to the N-terminal tryptophan of a cyclic 13-amino acid peptide (FIG. 1). Daptomycin has an excellent profile of activity because it is highly effective against most gram-positive bacteria; it is highly bactericidal and fast-acting; it has a low resistance rate and is effective against antibiotic-resistant organisms. The compound is currently being developed in a variety of formulations to treat serious infections caused by bacteria, including, but not limited to, methicillin-resistant Staphylococcus aureus (MRSA) and vancomycin-resistant enterococci (VRE).

A number of United States Patents describe A-21978C₀ antibiotics and daptomycin-related lipopeptides including daptomycin (LY 146032). These patents also describe methods of producing and isolating the A-21978C₀ antibiotics and daptomycin-related lipopeptides.

U.S. Pat. Nos. RE32,333, RE32,455, 4,800,157, 4,874,843, and 4,885,243 describe methods of synthesizing and isolating daptomycin from fermentation cultures of Streptomyces roseosporus. U.S. Pat. Nos. RE32,310, RE32,311, 4,537,717, 4,482,487 and 4,524,135 describe A-21978C₀ antibiotics and methods of deacylating the A-21978C₀ antibiotic and reacylating the peptide nucleus and antibiotic derivatives made by this process. U.S. Pat. No. 5,912,226 (hereafter the '226 patent) describes the identification and isolation of two impurities produced during the manufacture of daptomycin, anhydro-daptomycin and the β-isomer form of daptomycin. None of these United States patents discloses a method for precipitating or crystallizing a lipopeptide in a manner to increase purity of the lipopeptide.

U.S. Pat. No. 4,439,425 (hereafter the '425 patent) discloses a crystalline lipopeptide and a method of crystallizing the lipopeptide. The lipopeptide disclosed in the '425 patent is structurally dissimilar from daptomycin and daptomycin-related lipopeptides. U.S. Pat. No. 5,336,756 (hereafter the '756 patent) also discloses a crystalline cyclic lipopeptide comprising a hexapeptide. The crystalline cyclic lipopeptide disclosed in the '756 patent is also structurally dissimilar from daptomycin and daptomycin-related lipopeptides. The '756 patent discloses that the lipopeptide, an echinocandin-type compound, can be obtained when aqueous n-propanol is employed as the crystallizing solvent. See, e.g., cols. 1-2 of the '756 patent. Neither the '425 patent nor the '756 patent disclose methods of crystallizing or precipitating daptomycin or a daptomycin-related lipopeptide, nor do they disclose methods of crystallizing or precipitating lipopeptides produced by Streptomyces.

It would be advantageous to develop a method of crystallizing or precipitating daptomycin to provide an improved purification method for this compound. In addition, a crystalline or highly purified precipitated form of daptomycin would be useful in formulating pharmaceutical compositions for treating bacterial infections. Further, a crystalline or highly purified precipitated form of daptomycin would be useful in a method to make a sterile product, particularly bulk sterile product. Thus, there is a need for methods to produce crystalline or precipitated daptomycin. However, there has been no simple and robust method that has been effective in crystallizing or precipitating daptomycin that results in a daptomycin that is more pure after crystallization or precipitation than before.

### SUMMARY OF THE INVENTION

The instant invention addresses these problems by providing methods for crystallizing or precipitating daptomycin. In one embodiment, the invention provides methods for crystallizing daptomycin. In another embodiment, the methods provide daptomycin that is more pure after crystallization or precipitation than before crystallization or precipitation.

The invention also provides robust processes for producing and purifying daptomycin comprising, inter alia, crystallizing or precipitating daptomycin. In one embodiment, the crystallizing or precipitating steps of the processes are used to purify the daptomycin. In another embodiment, the processes are used for large-scale and/or commercial production of daptomycin.

### BRIEF DESCRIPTION OF THE DRAWINGS

FIG. 1 shows the structure of daptomycin.

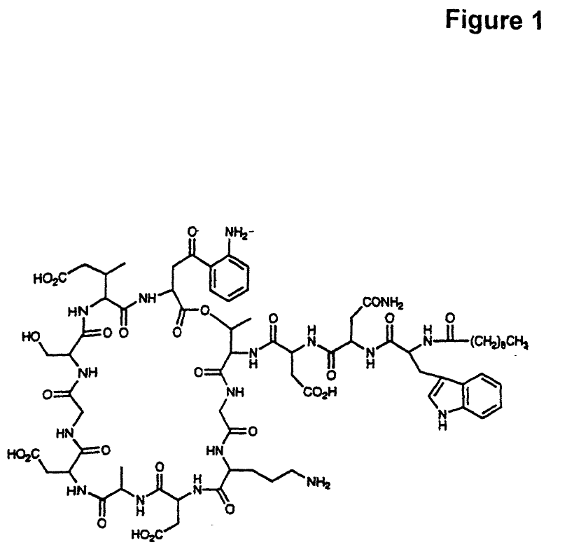

FIG. 2 shows a photomicrograph of urchin-like crystal or crystal-like particle of daptomycin produced by the method described in Example 12.

FIG. 3 shows a photomicrograph of needle-like crystals of daptomycin.

FIG. 4 shows a photomicrograph of rod-like crystals of daptomycin.

FIG. 5 shows photomicrographs of daptomycin samples at 100× magnification. Photomicrographs of amorphous daptomycin are shown using plane transmitted light (A) and using crossed polarized light (B). Photomicrographs of daptomycin crystals are shown using plane transmitted light (C and E) and using crossed polarized light (D and F). The daptomycin crystals were produced by the protocol disclosed in Example 7.

FIG. 6 shows an x-ray powder diffraction pattern for amorphous daptomycin.

FIG. 7 shows an x-ray powder diffraction pattern for a daptomycin crystal produced by the protocol described in Example 7.

FIG. 8 shows an x-ray powder diffraction pattern for a second sample of a daptomycin crystal produced by the protocol described in Example 7.

FIG. 9 shows birefringence of a crystal-like particle of daptomycin when exposed to polarized light. The crystal-like particle was produced by the method described in Example 12.

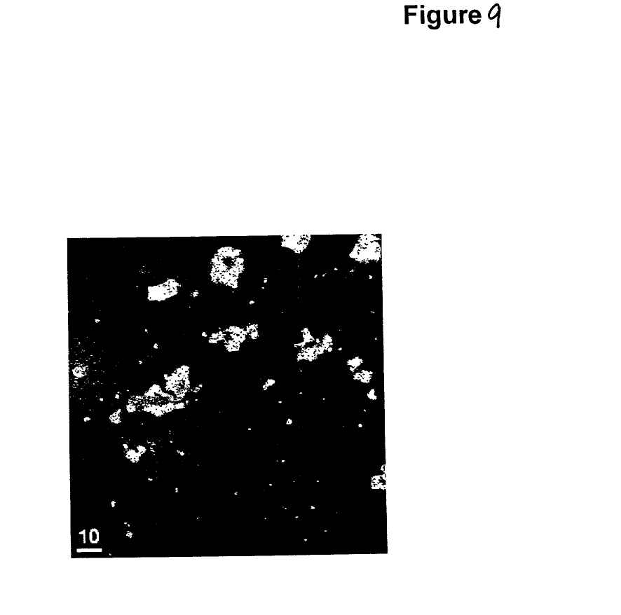

### DETAILED DESCRIPTION OF THE INVENTION

Objects of the Invention

One object of the present invention is to provide methods for crystallizing or precipitating daptomycin. In another embodiment, the methods increase the purity of the daptomycin compared to the purity of the daptomycin prior to crystallization or precipitation. The methods comprise the steps of providing an amorphous preparation of daptomycin and crystallizing or precipitating the daptomycin under conditions in which the crystalline or precipitated, crystal-like daptomycin is more pure than the amorphous preparation of the daptomycin. In one embodiment, the amorphous preparation is no greater than 92% pure and the crystalline or crystal-like daptomycin purified therefrom is at least 95% pure, and may be at least 96%, 97% or 98% or more pure. In another embodiment, the amorphous preparation is no greater than 80% pure and the crystalline or crystal-like daptomycin purified therefrom is at least 95% pure, and may be at least 96%, 97% or 98% or more pure. In another embodiment, the amorphous preparation is no greater than 60% pure and the crystalline or crystal-like daptomycin purified therefrom is at least 95% pure, and may be at least 96%, 97% or 98% ore more pure. In yet another embodiment, the amorphous preparation is no greater than 40% pure and the crystalline or crystal-like daptomycin purified therefrom is at least 95% pure, and may be at least 96%, 97% or 98% or more pure. In another embodiment, the amorphous preparation is no greater than 20% pure and the crystalline or crystal-like daptomycin purified therefrom is at least 95% pure, and may be at least 96%, 97% or 98% or more pure. In a further preferred embodiment, the amorphous preparation is no greater than 10% pure and the crystalline or crystal-like daptomycin purified therefrom is at least 95% pure, and may be at least 96%, 97% or 98% or more pure.

Another object of the invention is to provide processes for making and purifying a daptomycin comprising, inter alia, crystallizing or precipitating the daptomycin. In one embodiment, the crystallizing or precipitating steps are used to purify the daptomycin. In a preferred embodiment, the crystallization or precipitation is performed by batch crystallized or precipitation. In another embodiment, the process is a large-scale process for commercial production of daptomycin. In one embodiment, the daptomycin is produced by fermentation.

The fermentation product is then purified by a variety of purification techniques including crystallization or precipitation. In one embodiment, the crystallization or precipitation step may be used in combination with other purification techniques including microfiltration, size exclusion ultrafiltration and/or anion exchange chromatography. In one embodiment, the crystallization or precipitation step is used to replace one or more purification techniques that is used in a purification process that does not use crystallization or precipitation. In another embodiment, the crystallization or precipitation step is used to increase purification compared to the other steps without the crystallization or precipitation step. In a preferred embodiment, the method comprises a step of collecting the crystalline or crystal-like daptomycin after crystallization or precipitation.

The methods described herein can be used to produce crystalline or crystal-like form of daptomycin having any crystalline or crystal-like shape including urchin-like (cluster of needles joined together to visually resemble a sea urchin), needle-like, rod-like, plate-like or flake-like. In one embodiment, the methods can be used to provide crystalline or crystal-like daptomycin having a purity of at least 80%, and may be at least 85%, 90% pure. In another embodiment, the methods can be used to provide a crystalline or crystal-like form of the daptomycin having a purity of at least 95%, and may be at least 96%, 97%, 98% or more.

Definitions

Unless otherwise defined, all technical and scientific terms used herein have the meaning as commonly understood by one of ordinary skill in the art to which this invention belongs. The practice of the present invention employs, unless otherwise indicated, conventional techniques of chemistry, biochemistry, biophysics and microbiology and basic terminology used therein.

A “daptomycin-related molecule” includes, inter alia, daptomycin, A54145 or other lipopeptide that is structurally related to daptomycin, such as a daptomycin-related lipopeptide, including all stereoisomers that may be made at any chiral centers present in these molecules.

A “daptomycin-related lipopeptide” includes, without limitation, a lipopeptide disclosed in U.S. Pat. Nos. 4,537,717, 4,482,487, RE32,31 1, RE32,310, and 5,912,226, currently in reissue as U.S. application Ser. No. 09/547,357. Daptomycin-related lipopeptides also include those disclosed in International PCT Publication WO 01/44272, published Jun. 21, 2001; International PCT Publication WO 01/44274, published Jun. 21, 2001; and International PCT Publication WO 01/44271, published Jun. 21, 2001; all of these applications are specifically incorporated herein by reference. The daptomycin-related lipopeptides disclosed in the above-identified applications relate to synthetic and semisynthetic lipopeptides in which the ornithine and/or kynurine residues, and/or the fatty acid side chain of daptomycin, are modified. Daptomycin-related lipopeptides further include an A-21978C₀ antibiotic in which the n-decanoyl fatty acid side chain of daptomycin is replaced by a n-octanoyl, n-nonanoyl, n-undecanoyl, n-dodecanoyl, n-tridecanoyl or n-tetradecanoyl fatty acid side chain.

The term “daptomycin” refers to the n-decanoyl derivative of the factor A-21978C₀-type antibiotic that contains an α-aspartyl group. “Daptomycin” is synonymous with LY 146032.

The term “anhydro-daptomycin” refers to a daptomycin-related lipopeptide in which an α-aspartyl group of daptomycin is cyclized to a succinimido group. See, e.g., the '226 patent for the structure of anhydro-daptomycin.

The term “P-isomer” or “P-isomer of daptomycin” refers to a daptomycin-related lipopeptide that contains a β-aspartyl group instead of an α-aspartyl group. See, e.g., the '226 patent for the structure of β-isomer of daptomycin.

The term “isolated” refers to a compound or product that is at least 5%, 10%, 20%, 30%, 40%, 50%, 60%, 70%, 80% or 90% of the compound present in a mixture. It will be understood that the term “isolated” also refers to a compound that is at least 5-10%, 10-20%, 20-30%, 30-40%, 40-50%, 50-60%, 60-70%, 70-80% or 80-90% of the compound present in the mixture group. The percentage of compound in a mixture may be measured by any means known in the art, as described below for measuring purity of a compound.

“Substantially pure” refers to a sample having at least 95% of a desired compound. Preferably, daptomycin is “substantially pure” when at least 95% to at least 97% of a sample is daptomycin.

Daptomycin or a daptomycin-related lipopeptide is “essentially pure” when at least 98% to at least 99% of a sample is daptomycin or a daptomycin-related lip opeptide, respectively.

Daptomycin or a daptomycin-related lipopeptide is “substantially free” of another compound when the other compound is present in an amount that is no more than 1% of the amount of the daptomycin or the daptomycin-related lipopeptide preparation, respectively.

Daptomycin or a daptomycin-related lipopeptide is “essentially free” of another compound when the other compound is present in an amount that is no more than 0.5% of the amount of the daptomycin or the daptomycin-related lipopeptide preparation, respectively.

Daptomycin or a daptomycin-related lipopeptide is “free” of another compound when the other compound is present in an amount that is no more than 0.1% of the amount of the daptomycin or the daptomycin-related lipopeptide preparation, respectively. Alternatively, daptomycin or a daptomycin-related lipopeptide is “free” of another compound when the compound cannot be detected by HPLC under conditions of maximum sensitivity in which a limit of detection is approximately 0.05% or less of the amount of the daptomycin or the daptomycin-related lipopeptide preparation, respectively.

“Purified” daptomycin refers to substantially pure daptomycin, essentially pure daptomycin, or a salt thereof, or to daptomycin or a salt thereof which is substantially free, essentially free, or free of another compound. Similarly, a “purified” daptomycin-related lipopeptide refers to a substantially pure daptomycin-related lip opeptide, an essentially pure daptomycin-related lipopeptide, or a salt thereof, or to a daptomycin-related lipopeptide or a salt thereof which is substantially free, essentially free, or free of another compound.

“Crude” daptomycin refers to daptomycin or a salt thereof that is less than 90% pure. Similarly, “crude” daptomycin-related lipopeptide refers to a daptomycin-related lipopeptide or a salt thereof that is less than 90% pure.

“Semi-purified” daptomycin refers to daptomycin or a salt thereof that is at least 90% pure and less than 95% pure. Similarly, “semi-purified” daptomycin-related lipopeptide refers to a daptomycin-related lipopeptide or a salt thereof that is at least 90% pure and less than 95% pure.

The purity of daptomycin or daptomycin-related lipopeptide refers to the daptomycin prior to its formulation in a pharmaceutical composition. The purity of the daptomycin is referred to by “percent purity.” The measure of purity is not a measure of degree of crystallinity of the crystalline preparation. The purity may be measured by any means including nuclear magnetic resonance (NMR), gas chromatography/mass spectroscopy (GC/MS), liquid chromatography/mass spectroscopy (LC/MS) or microbiological assays. One preferred means for measuring the purity of daptomycin is by analytical high pressure liquid chromatography (HPLC). Two methods of analytical HPLC are described in International PCT Publication WO 01/53330, published Jul. 26, 2001, which is herein incorporated specifically by reference.

A “daptomycin crystal” refers to one or more crystals of daptomycin or of a daptomycin salt. The determination of daptomycin as a crystal can be determined by any means including, inter alia, optical microscopy, electron microscopy, x-ray powder diffraction, solid state nuclear magnetic resonance (NMR) or polarizing microscopy. Microscopy can be used to determine the crystal length, diameter, width, size and shape, as well as whether the crystal exists as a single particle or is polycrystalline.

Daptomycin is “crystal-like” if it is determined to have crystalline characteristics when determined by one means, e.g., visually or by optical or polarizing microscopy, but does not have crystalline characteristics when determined by another means, e.g., x-ray powder diffraction. Daptomycin that is “crystal-like” may be crystalline under certain conditions but may become non-crystalline when subjected to other conditions.

“Crystalline daptomycin” or a “crystalline form of daptomycin” refers to a preparation of daptomycin or salt thereof that comprises daptomycin crystals. In one embodiment, a crystalline daptomcyin may comprise some amount of amorphous daptomycin. In one embodiment, the crystalline daptomycin produced by the methods described herein comprises more than 50% by weight of the daptomycin crystals. In another embodiment, the crystalline daptomycin comprises more than 60%, 70%, 80%, 90% or 95% of the daptomycin crystals. The crystalline daptomycin may comprise 50-60%, 60-70%, 70-80%, 80-90% or 90-95% of the daptomycin crystals. In another embodiment, the crystalline daptomycin comprises more than 95% of the daptomycin crystals, e.g., at least 96%, 97%, 98% or 99% daptomycin crystals or 100% daptomycin crystals. The crystalline daptomycin may also comprise anywhere from 95-100% daptomycin crystals.

An “amorphous” form of daptomycin refers to daptomycin that comprises few or no crystals or crystal-like daptomycin (or crystal-like particles) as defined herein. In one embodiment, an amorphous daptomycin comprises less than 20% by weight of daptomycin crystals or crystal-like daptomycin. In another embodiment, an amorphous daptomycin comprises less than 10% by weight of daptomycin crystals or crystal-like daptomycin. In another embodiment, an amorphous daptomycin comprises less than 5% by weight of daptomycin crystals or crystal-like daptomycin. In a still further preferred embodiment, an amorphous daptomycin comprises less than 1% by weight of daptomycin crystals or crystal-like daptomycin.

“Batch crystallization” refers to a method in which daptomycin is mixed with the crystallization reagents in solution and the daptomycin is allowed to crystallize in solution. “Batch precipitation” refers to a method in which the daptomycin is mixed with precipitation reagents in solution and the daptomycin is allowed to precipitate in solution. In one embodiment, the crystalline or precipitated preparation is collected from the solution. In another embodiment, the crystalline or precipitated preparation is collected by filtration or centrifugation.

“Organic precipitant” refers to a polyethylene glycol (PEG) or polyethylene glycol monomethyl ether (PEG MME) or compounds that are chemically similar.

“Salts” refer to ionic compounds. These ionic compounds may act as precipitants.

“Low molecular weight alcohols” are organic compounds containing at least one alcohol functional group, and eight carbon atoms or less. For example, low molecular weight alcohols include, without limitation, methanol, isopropanol, and tert-butanol.

“Polyhydric alcohols” refer to compounds that contain more than one alcohol group, and less than eight carbon atoms. Polyhydric alcohols, for example, include, without limitation, 1,6 hexanediol, ethylene glycol, propylene glycol, glycerol, 1,2-propanediol, 2-methyl-2,4-pentanediol and 1,4 butanediol.

Methods for Producing Purified Daptomycin

One object of the invention is to provide a method for purifying daptomycin comprising the steps of providing an amorphous preparation of daptomycin and crystallizing or precipitating the daptomycin. In one embodiment, the daptomycin has a higher degree of purity after crystallization or precipitation than prior to being subjected to crystallization or precipitation. Daptomycin may be crystallized by hanging drop, sitting drop or sandwich drop vapor diffusion, liquid-liquid or free interface diffusion, microdialysis or dialysis, slow solvent evaporation, sublimation, or microbatch or batch crystallization. In general, daptomycin may be precipitated in a similar way, preferably daptomycin is precipitated by batch precipitation.

Daptomycin may be crystallized or precipitated following the teachings of this specification. In one embodiment, daptomycin can be crystallized or precipitated by providing a solution comprising daptomycin with a low molecular weight or polyhydric alcohol, a pH buffering agent and a salt comprising a monovalent or divalent cation and allowing precipitation or crystallization to occur, as discussed further infra. In another embodiment, the salt has buffering capacity such that an additional pH buffering agent does not have to be present in the solution. In another embodiment, the salt comprises a divalent cation. In a preferred embodiment, the solution provided does not include PEG or PEG-MME or chemically similar compounds. In an embodiment, the method for precipitating or crystallizing daptomycin generally comprises the steps of:

a) mixing daptomycin with a salt comprising a monovalent or divalent cation, an optional pH buffering agent and a low molecular weight or polyhydric alcohol; and

b) allowing the daptomycin to precipitate or crystallize from the solution under the appropriate temperature conditions.

The samples may be monitored, inter alia, for crystal or precipitate formation by microscopic examination and the yield may be followed spectrophotometrically.

In another embodiment, daptomycin can be crystallized by providing a solution comprising a low molecular weight or polyhydric alcohol(s), salts and an organic precipitant as discussed further infra. In general, for batch crystallization, daptomycin is dissolved in a solution and low molecular weight alcohols, salts, buffers and/or organic precipitants are added to the solution. The samples are then crystallized under the appropriate temperature conditions, with or without stirring. The samples may be monitored, inter alia, for crystal formation by microscopic examination and the yield may be followed spectrophotometrically.

As discussed above, daptomycin is crystallized or precipitated in the presence of one or more alcohols. In a preferred embodiment, the alcohol is a low molecular weight or polyhydric alcohol. Examples of low molecular weight or polyhydric alcohols include, without limitation, methanol, isopropanol, tert-butanol, 1,6 hexanediol, ethylene glycol, propylene glycol, glycerol, 1,2-propanediol, 2-methyl-2,4-pentanediol and 1,4 butanediol. In a preferred embodiment, the alcohol is isopropanol, tert-butanol, glycerol, 1,6-hexanediol, 1,2-propanediol, 1,4-butanediol, propylene glycol and/or ethylene glycol. In a more preferred embodiment, the alcohol is isopropanol.

Salts include, inter alia, magnesium or sodium formate, ammonium sulfate, ammonium dihydrogen phosphate, calcium acetate, zinc acetate, tri-sodium citrate dihydrate, magnesium acetate, sodium acetate, magnesium chloride, cadmium chloride, ammonium acetate, sodium chloride and lithium sulfate. In one embodiment, the salt comprises a monovalent cation, e.g., sodium. In a preferred embodiment, the salt comprises a divalent cation. In an even more preferred embodiment, the salt comprises a calcium cation, a magnesium cation or a manganese cation. In a further preferred embodiment, the salt comprises a calcium divalent cation. In one embodiment, the salt is calcium chloride, calcium acetate, zinc acetate, sodium citrate, tri-sodium citrate dihydrate, magnesium chloride, lithium sulfate, sodium chloride, magnesium acetate, sodium acetate or a manganese salt, such as manganese acetate or manganese chloride. In a preferred embodiment, the salt is calcium acetate. Examples of other salts that comprise a divalent cation, such as a calcium cation, are known in the art, and include, inter alia, those listed in the 2000 Sigma catalog, herein incorporated by reference. Without wishing to be bound to any theory, it is thought that the salt cation may neutralize the negative charges on the four carboxylic acids of daptomycin. Organic precipitants include, inter alia, polyethylene glycols (PEGs) that can vary in average molecular weight from between 300 and 10,000, or polyethylene glycol monomethyl ether (PEG-MME). In a preferred embodiment, the organic precipitant is PEG 300, PEG 600, PEG 2000, PEG 4000, PEG 8000 or PEG 10,000.

Daptomycin is precipitated or crystallized from a solution that is buffered to pH 5.0 to 9.5. In one embodiment, prior to being buffered, the solution has a pH of about 1.5, 2.0 or 3.0. In one embodiment, daptomycin is precipitated or crystallized from a solution of approximately pH 5.5 to approximately pH 7.5. In another embodiment, the buffer has a pH of approximately 5.9 to approximately pH 6.3. In one embodiment, the buffered solution may be obtained by using a pH buffering agent. Examples of pH buffering agents include, without limitation, Tris, phosphate, citrate, HEPES, CHES, sodium acetate or 2-morpholinoethanesulfonic acid (MES), sodium borate, sodium cacodylate, imidazole and tri-sodium citrate dihydrate. In a preferred embodiment, the salt is sodium cacodylate, sodium acetate, tri-sodium citrate dihydrate, HEPES, MES, CHES, imidazole, calcium acetate and Tris-HCl. In a more preferred embodiment, the pH buffer is calcium acetate pH 6. 1, sodium acetate pH 6.1, sodium cacodylate pH 6.5, tri-sodium citrate dihydrate pH 5.6, HEPES pH 7.5, imidazole pH 8, MES pH 6.0, calcium acetate pH 6 and Tris-HCl pH 8.5. In another embodiment, the solution may be buffered by using a salt that also has buffering capacity. In a preferred embodiment, the pH buffer is calcium acetate pH 6.1.

Daptomycin is precipitated or crystallized using hanging drop vapor diffusion from a solution containing 2 to 40% low molecular weight or polyhydric alcohol, 0.001 to 0.5 M salt and 0.005 to 0.2 M pH buffering agent. In a preferred embodiment, the daptomycin is precipitated or crystallized from a solution containing 3 to 30% low molecular weight or polyhydric alcohol, 0.01 to 0.3 M salt and 0.01 to 0.1 M pH buffering agent. In a more preferred embodiment, the daptomycin is precipitated or crystallized from a solution containing 5 to 20% low molecular weight or polyhydric alcohol, 0.02 to 0.1 M salt and 0.02 to 0.07 M pH buffering agent. The solution provided may or may not include polyethylene glycol (PEG) or polyethylene glycol monomethyl ether (PEG-MME).

The daptomycin is precipitated or crystallized using batch crystallization from a solution containing 65 to 95% low molecular weight or polyhydric alcohol, 0.001 to 0.5 M salt and 0.001 to 0.2 M pH buffering agent. In a preferred embodiment, the daptomycin is precipitated or crystallized from a solution containing 70 to 90% low molecular weight or polyhydric alcohol, 0.005 to 0.04 M salt and 0.005 to 0.04 M pH buffering agent. In some embodiments, the daptomycin is crystallized from a solution which also comprises 3-8% organic precipitant. In a more preferred embodiment, the daptomycin is precipitated or crystallized from a solution containing 80 to 85% low molecular weight or polyhydric alcohol, 0.01 to 0.03 M salt and 0.01 to 0.03 M pH buffering agent. In some embodiments, the solution further comprises about 4 to 5% organic precipitant, e.g., PEG or PEG-MME. In other embodiment, the solution provided does not include polyethylene glycol (PEG) or polyethylene glycol monomethyl ether (PEG-MME).

The daptomycin is precipitated or crystallized at a temperature from approximately 0° C. to approximately 30° C. to obtain precipitate or crystal formation, respectively. In a preferred embodiment, daptomycin is crystallized or precipitated at a temperature of approximately 20-30° C. In a more preferred embodiment, the mixture is crystallized or precipitated at approximately 23-28° C. In an even more preferred embodiment, the mixture is crystallized or precipitated at approximately 27° C. The mixture may be crystallized or precipitated for any time period that results in crystallization or precipitation, preferably approximately one hour to approximately two weeks. In a preferred embodiment, the mixture is stored for a period of approximately three hours to approximately 24 hours, more preferably approximately 8-18 hours.

The methods described herein can be used to provide daptomycin having a shape that is, without limitation, needle-like, rod-like, urchin-like, flake-like, plate-like or clusters thereof. In one embodiment, the methods provide daptomycin crystals or crystal-like particles are urchin-like, rod-like or needle-like. The shape of the crystal or crystal-like particle may be determined, inter alia, by optical or electron microscopy. In another embodiment, daptomycin crystals or crystal-like particles produced may be any size that is at least approximately 0.5 μm in diameter in any one dimension. In a more preferred embodiment, daptomycin crystals or crystal-like particle are at least 5 μm, more preferably at least 10 μm. In an even more preferred embodiment, the daptomycin crystals or crystal-like particles are at least 50 μm, more preferably at least 100 μm. The size of the crystal maybe determined by any method known to one having ordinary skill in the art. See, e.g., United States Pharmacopeia (USP), pp. 1965-67.

The properties of a crystalline or crystal-like daptomycin may be determined by any method known to one having ordinary skill in the art. The properties that can be determined include the crystalline or crystal-like daptomycin's size, shape, birefringence properties, powder x-ray diffraction properties, solid state NMR properties, melting temperature and stability to heat, light, humidity, and degradation. In a preferred embodiment, one having ordinary skill in the art may determine whether daptomycin is crystalline by powder x-ray diffraction. Powder x-ray diffraction is highly useful for determining whether a preparation is crystalline when the sample is a randomly-oriented collection of small crystals. Diffraction by a mass of randomly-oriented microcrystals produces a series of lines or rings (dependent of the detector) characteristic of the molecule studied and its structure. In a preferred embodiment, powder diffraction is measured by an Automated Powder Diffraction instrument in order to determine whether daptomycin is crystalline. See, e.g., Atkins et al., Physical Chemistry, pp. 710-716 (1978), herein incorporated by reference for a discussion of the Debye-Scherrer method for powder diffraction. Any powder diffractometer instrument known in the art that is equipped with any detector for powder diffraction that known in the art could be used to measure the diffraction pattern.

In a preferred embodiment of the invention, daptomycin is crystallized or precipitated using a buffering agent between approximately pH 5.0 and 9.5, a salt and an alcohol at a temperature of approximately 24-28° C. for a period of approximately three to 24 hours. In a preferred embodiment, the salt is a buffering agent and comprises a divalent cation and the alcohol is a low molecular weight alcohol, and the pH is between approximately pH 5.5 and 7.5. In an even more preferred embodiment, the salt is a calcium salt, the alcohol is isopropanol and the pH is between approximately pH 5.9 and 6.3. In embodiments were the solution includes an organic precipitant, preferably the organic precipitant is PEG 4000 or PEG 8000. In another embodiment, the daptomycin is precipitated or crystallized from a solution containing 12 to 18% glycerol, 0.3 to 0.8 m salt, 0.03 to 0.08 m pH buffering agent, and 12-18% PEG 600. Examples 2-3 provide methods for precipitating a highly pure crystal-like daptomycin. One having ordinary skill in the art, following the teachings of the instant specification, may modify the crystallization/precipitation conditions provided in the examples to crystallize or precipitate daptomycin. Further, although the teachings of the instant specification describe the use of a single crystallization or precipitation step in a process for purifying daptomycin, one having ordinary skill in the art following the teachings of the specification may use multiple crystallization or precipitation steps in a process for purifying daptomycin. It may be advantageous to employ multiple rounds of crystallization or precipitation as disclosed herein in order to further increase purity of daptomycin.

After crystallization or precipitation, one may collect the crystalline material or crystal-like precipitate by any method known in the art. In a preferred embodiment, the crystalline material or crystal-like precipitate is collected by centrifugation or filtration. In an even more preferred embodiment, the crystalline material or crystal-like precipitate is collected by filtration because filtration is easily incorporated into a large-scale process for producing daptomycin. After the crystalline material or crystal-like precipitate is collected, it may be washed to remove excess crystallizing or precipitating reagents. Any wash solvent known in the art may be chosen so long as it does not appreciably dissolve the crystalline material or crystal-like precipitate. An example of a wash solvent is provided in Example 12. After the crystalline material or crystal-like precipitate is washed, it may be dried by any method known in the art. Examples of drying methods include air-drying, lyophilization (freeze-drying) or desiccation. In a preferred method, the crystalline material or crystal-like precipitate is desiccated. See, e.g., Example 12. In another embodiment, the crystalline daptomycin's stability may be determined by its residual antibiotic activity or its degradation. The antibiotic activity may be measured in a standard agar-diffusion assay against various bacterial strains. See, e.g., Example 32 of U.S. Pat. No. 4,537,717, specifically incorporated herein by reference. The amount of degradation can be measured by, inter alia, HPLC analysis, such as that described in International PCT Publication WO 01/53330, published Jul. 26, 2001. In a preferred embodiment, the stability of the crystalline daptomycin is greater than that of the amorphous form of the daptomycin. The stability of the crystalline daptomycin may be determined by exposing the crystalline daptomycin and an amorphous form thereof to heat, light, humidity, and measuring the degree of degradation of the crystalline form to that of the amorphous form.

Degradation of daptomycin may be measured by determining the biological activity of daptomycin or any applicable physical parameter. In one embodiment, degradation may be measured by determining a particular biological activity of daptomycin after it has been subjected to heat, light, humidity, changes in pH or extreme pH, and comparing it to the same biological activity of daptomycin prior to any tests of stability. The amount of degradation may be determined, for example, by determining the percentage of biological activity remaining after the test of stability. The percentage of remaining biological activity may be compared to that of an amorphous form of daptomycin that has been subjected to the same test. In one embodiment, the crystalline daptomycin may be tested for its antibiotic activity, e.g., the amount of antibiotic activity of the daptomycin against gram-positive bacteria, both prior to and after a test of its stability and compared to an amorphous form that has been tested prior to and after a degradation test.

Degradation of daptomycin may also be measured by a physical assay. In one embodiment, degradation may be measured by determining the percentage of intact crystalline daptomycin that remains after a test of its stability. The percentage of remaining intact daptomycin may be compared to that of an amorphous form of daptomycin that has been subjected to the same test for stability. In a preferred embodiment, the degradation of daptomycin may be measured by HPLC, ultraviolet spectroscopy, infrared spectroscopy, NMR, or mass spectroscopy. In an even more preferred embodiment, HPLC is used to determine the percentage of intact daptomycin that remains after a crystalline form of daptomycin has been subjected to a test of its stability.

Without wishing to be bound by any theory, applicants believe that daptomycin is crystallized by the methods described above. However, it is thought that washing and/or drying the daptomycin crystals causes the daptomycin crystalline material to revert to a non-crystalline but still crystal-like form. Nevertheless, even if the methods described above only precipitate rather than crystallize the daptomycin, the methods still are advantageous because the methods purify daptomycin.

In one embodiment, the method provides a crystalline or crystal-like daptomycin comprising a lower amount of one or more impurities compared to daptomycin before crystallization or precipitation. In one embodiment, the method produces a crystalline or crystal-like daptomycin comprising a lower level of anhydro-daptomycin and/or the β-isomer of daptomycin compared to daptomycin before crystallization or precipitation. In another embodiment, the method provides crystalline or crystal-like daptomycin comprising a lower level of all impurities compared to amorphous daptomycin.

The crystalline or crystal-like daptomycin produced by the method described above likely comprises monovalent or divalent cations and water. In a preferred embodiment, the crystalline or crystal-like daptomycin comprises a divalent cation. In a more preferred embodiment, the divalent cation is a calcium cation. In an even more preferred embodiment, the crystalline or crystal-like daptomycin comprises approximately 1-10% by weight of a divalent calcium cation and approximately 0-15% by weight of water as determined by atomic absorption or thermal gravity analysis. In a further preferred embodiment, the crystalline or crystal-like daptomycin comprises approximately 5% by weight of a divalent calcium cation and approximately 10% by weight of water; by HPLC analysis, the purity of the crystalline or crystal-like daptomycin is at least 95%, 96%, 97% or 98% or is any purity between 95-98%, relative to related substances and organic contaminants. Alternatively, the crystalline or crystal-like daptomycin comprises a monovalent cation such as sodium. Without wishing to be bound by any theory, it is thought that daptomycin may form a salt with the monovalent or divalent cation when it crystallizes or precipitates.

The crystalline form of daptomycin may exhibit an increased solubility in a solution or an increased rate of reconstitution in a solution than an amorphous form of the daptomycin. One may measure whether the crystalline daptomycin exhibits an increased solubility or increased reconstitution rate by any method known in the art. For instance, one may dissolve a defined amount of crystalline daptomycin in an aqueous solution and measure the concentration of the dissolved daptomycin and compare it to the concentration of dissolved daptomycin that has been prepared by dissolving the same amount of amorphous daptomycin in an aqueous solution. Similarly, one may measure the reconstitution rate of a crystalline daptomycin by adding the crystalline daptomycin to an aqueous solution and then measuring the concentration of dissolved daptomycin over time and comparing it to the reconstitution rate of amorphous daptomycin that has been measured in the same way. The concentration of daptomycin is measured by HPLC.

The methods described above provide for the production of crystalline or crystal-like daptomycin that is more pure than the amorphous daptomycin from which it is crystallized or precipitated. In another embodiment, daptomycin has a purity of no more than 92% before crystallization and has a purity of at least approximately 95%, 96%, 97% or 98% purity, or any purity between 95-98%, after crystallization or precipitation as a crystalline or crystal-like form. In a still further preferred embodiment, daptomycin has a purity of no more than 90% before crystallization and has a purity of approximately at least 97% or 98% after crystallization or precipitation.

In another embodiment, the daptomycin has a purity of no more than 80%, preferably no more than 70% and more preferably no more than 60% purity before crystallization or precipitation, and has at least approximately 95%, 96%, 97% or 98% purity, or any purity between 95-98%, after purification. In another embodiment, the daptomycin has a purity of no more than 50%, preferably no more than 40%, more preferably no more than 30% purity before crystallization and has at least approximately 95%, 96%, 97% or 98% purity, or any purity between 95-98%, after purification by crystallization or precipitation. Further preferred is an embodiment in which daptomycin has a purity of no more than 20%, more preferably no more than 15%, even more preferably no more than 10% purity before crystallization and has at least approximately 95%, 96%, 97% or 98% purity, or any purity between 95-98%, after purification.

A daptomycin preparation may be obtained by any method disclosed, e.g., in any one U.S. Pat. Nos. RE32,333, RE32,455, 4,800,157, RE32,310, RE32,311, 4,537,717, 4,482,487, 4,524,135, 4,874,843, 4,885,243 or 5,912,226, which are herein incorporated specifically by reference. A daptomycin preparation may also be obtained by one of the methods described in International PCT Publication WO 01/53330, published Jul. 26,2001. After the daptomycin preparation is prepared, the daptomycin preparation is crystallized or precipitated following the teachings of the specification described herein to produce a crystalline or crystal-like daptomycin that is more pure or that contains lower levels of specific impurities, e.g., anhydro-daptomycin, than the daptomycin preparation from which it is prepared.

Processes for Producing Daptomycin from Fermentation Cultures

In one embodiment, the purification method comprises fermenting Streptomyces roseosporus to obtain a fermentation culture containing daptomycin. In one embodiment, the S. roseosporus may be fermented as described in U.S. Pat. No. 4,885,243. In another embodiment, the fermentation conditions in which the A-21978C₀-containing crude product is produced by Streptomyces roseosporus is altered in order to increase daptomycin production and decrease impurities and related contaminants produced by the S. roseosporus fermentation culture as described in International PCT Publication WO 01/53330, published Jul. 26, 2001. The WO 01/53330 publication describes fermenting S. roseosporus as described in the '243 patent with the modification that the decanoic acid feed is kept at the lowest levels possible without diminishing the overall yield of the fermentation.

Alternatively, daptomycin may be obtained by fermenting a bacterial strain or other producing organism that recombinantly produces daptomycin. In one embodiment, the recombinant bacterial strain or other recombinant organism comprises the daptomycin biosynthetic gene cluster. In another embodiment, the daptomycin biosynthetic gene cluster or a portion thereof is introduced into the organism or bacterial strain via a bacterial artificial chromosome (BAC). In another embodiment, the recombinant bacterial strain used is S. roseosporus or S. lividans comprising a BAC containing the daptomycin biosynthetic gene cluster. U.S. Provisional Application No. 60/272,207, filed Feb. 28, 2001 describes the daptomycin biosynthetic gene cluster from S. roseosporus and uses thereof, and is hereby incorporated by reference in its entirety.

In order that this invention may be more fully understood, the following examples are set forth. These examples are for the purpose of illustration only and are not to be construed as limiting the scope of the invention in any way.

### EXAMPLE 1

Daptomycin was prepared by conventional techniques. The daptomycin preparation was a pale yellow amorphous powder, with a solubility at 25° C. of greater than 1 g/mL in water and a solubility of 2.8 mg/mL in ethanol. The amorphous daptomycin preparation was hygroscopic and decomposed at 215° C.

The remaining examples describe crystallizing or precipitating daptomycin in the presence or absence of an organic precipitant (e.g., PEG).

### EXAMPLE 2

In a microbatch crystallization, 25 μL of a daptomycin stock (20 mg/mL in methanol) was sequentially mixed with 15 μL of reagent stock (200 mM calcium acetate, 0.1 M cacodylate (pH 6.5), 18% [w/v] PEG 8000 and 15 μL ethylene glycol) to give a solution that was 27.5% aqueous component, 45% methanol and 27.5% ethylene glycol. Urchin-like crystals were formed at a yield of 50% with a purity of 98% as measured by HPLC.

### EXAMPLE 3

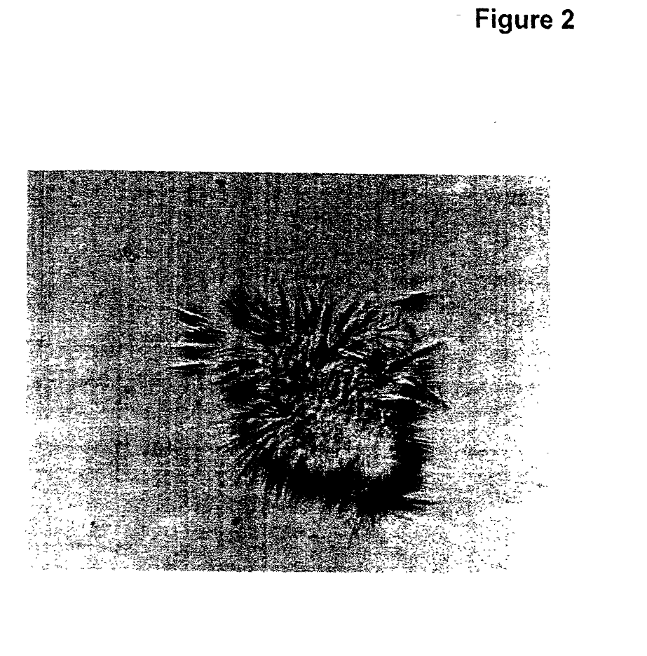

A daptomycin stock was prepared by dissolving 440 mg daptomycin in 1 mL of a buffer containing 25 mM sodium acetate (pH 5.0) and 5 mM CaCl₂. Crystallization was done by the vapor diffusion (hanging drop) method, in which 5 μL of the daptomycin stock was added to 5 μL of 0.1 M tri-sodium citrate dihydrate (pH 5.6), and 35% [v/v] tert-butanol in water to form a drop. The drop was suspended over a reservoir solution (0.1 M tri-sodium citrate dihydrate (pH 5.6), and 35% [v/v] tert-butanol in water) in an air-tight environment until crystallization occurred. This method yielded urchin-like daptomycin crystals. See, e.g., FIG. 2.

### EXAMPLE 4

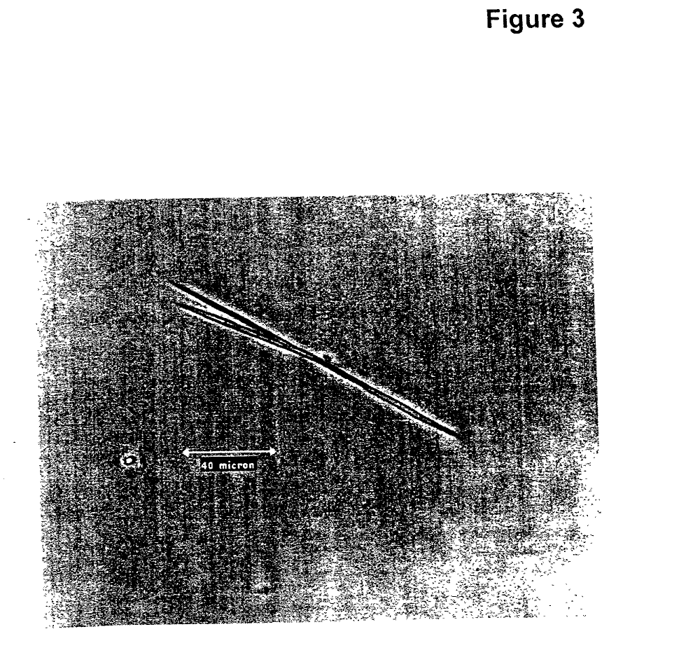

5 μL of a daptomycin stock prepared as in Example 3 was added to 5 μL of a solution containing 0. 1 M sodium cacodylate (pH 6.5), 0.2 M calcium acetate and 9% [w/v] PEG 8000. Crystallization was done by the vapor diffusion method as described in Example 3. This method yielded needle-like daptomycin crystals. See, e.g., FIG. 3.

### EXAMPLE 5

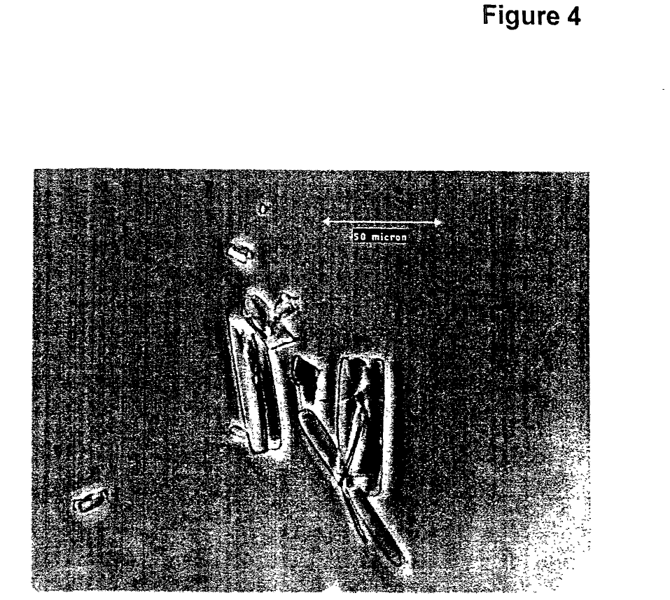

5 μL of a daptomycin stock prepared as in Example 3 was added to 5 μL of a solution of 0.1 M sodium cacodylate (pH 6.5), 0.2 M zinc acetate and 9% [w/v] PEG 8000 containing 0.1 μL benzamidine to give a final concentration of 220 mg/mL daptomycin. Crystallization was done by the vapor diffusion method as described in Example 3. This method yielded rod-like daptomycin crystals. See, e.g., FIG. 4.

### EXAMPLE 6

One mL of daptomycin (97.1% pure as determined by HPLC) at a concentration of 20-25 mg/mL in water was sequentially mixed with 231 μL water, 77 μL of calcium acetate (pH 6.0), 960 μL propylene glycol and 231 μL of 50% [w/v] PEG 4000. The solution was allowed to sit for 4-5 hours at 4° C. Urchin-like crystals were formed at a yield of 75%. The crystalline daptomycin was washed with isopropanol. The daptomycin was 98.4% pure as determined by HPLC.

### EXAMPLE 7

Daptomycin (200 mg, 97.1% pure) was dissolved in 2.54 mL water. The daptomycin solution was sequentially mixed in order with 10.0 mL methanol, 0.78 mL 1 M calcium acetate (pH 6.0), 9.50 mL propylene glycol and 2.20 mL 50% [w/v] PEG 4000 to give a final volume of 25.02 mL. The mixture was tumbled at room temperature for 10-14 hours in a hematology mixer (Fischer). Crystals began to appear within a few hours. Final yield was approximately 70-80% after 14 hours. The crystals were harvested by centrifugation at 1000 rpm for 15 minutes. The supernatant was removed and the crystals were resuspended in 12.5 mL isopropanol. The daptomycin suspension was transferred to a column (Biorad) and the isopropanol was removed by allowing it to drip by gravity. The crystals were dried by a nitrogen stream. Any lumps were broken up during the drying procedure to obtain a uniform dry sample. Crystals prepared by this method were urchin-like and had a purity of 98.37%.

### EXAMPLE 8

Daptomycin was crystallized according to Example 7 except that PEG 8000 was used in replacement of PEG 4000. The quantities of reagents used are identical to those in Example 7. Crystals prepared by this method were urchin-like and had a purity of 98.84%.

### EXAMPLE 9

Two daptomycin samples prepared according to Example 7 and one amorphous sample were analyzed for crystallinity using the USP <695> crystallinity test. Daptomycin particles were mounted in mineral oil on a glass slide and then were examined by polarizing light microscope (PLM). The particles were determined to be crystalline if they were birefringent (have interference colors) and had extinction positions when the stage was rotated.

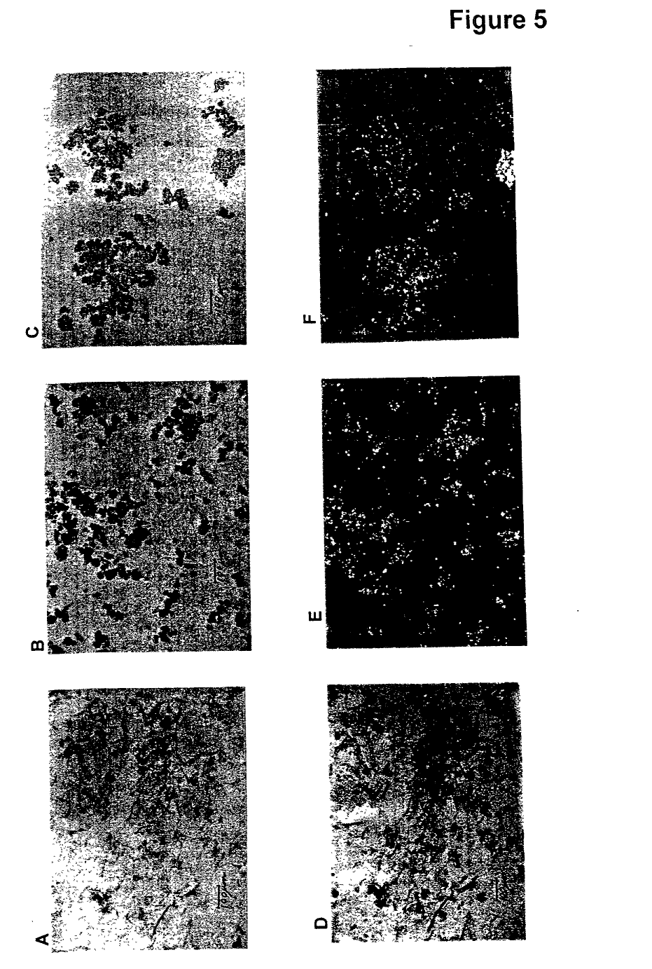

The amorphous daptomycin sample consisted of lacy, flaky particles that were not birefringent. There were a few sliver-like areas in some of the flakes that had weak birefringence, but the particles were primarily amorphous. In contrast, the daptomycin samples prepared according to Example 7 consisted of polycrystalline particles with weak birefringence and some extinction, indicating that they were primarily crystalline. See FIG. 5.

### EXAMPLE 10

Two daptomycin samples prepared according to Example 7 and one amorphous sample were analyzed for crystallinity by x-ray powder diffraction. The samples were analyzed on a Siemens D500 Automated Powder Diffractometer (ORS 1D No. LD-301-4), which was operated according to ORS Standard Operation Procedure EQ-27 Rev. 9. The diffractometer was equipped with a graphite monochromator and a Cu (λ=1.54 Å) x-ray source operated at 50 kV, 40 mA. Two-theta (θ) calibration is performed using an NBS mica standard (SRM675). The samples were analyzed using the following instrument parameters:

| | |
|---|---|
| Measuring Range for 2θ (degrees) | 4.0-40.0 |
| Step Width (degrees) | 0.05 |
| Measuring Time per Step (secs) | 1.2 |
| Beam Slits | 1(1°), 2(1°), 3(1°), 4(0.15°), 5(0.15°). |

Sample preparation was performed according to ORS Standard Operation Procedure MIC-7 Rev. 1 using a zero background sample plate.

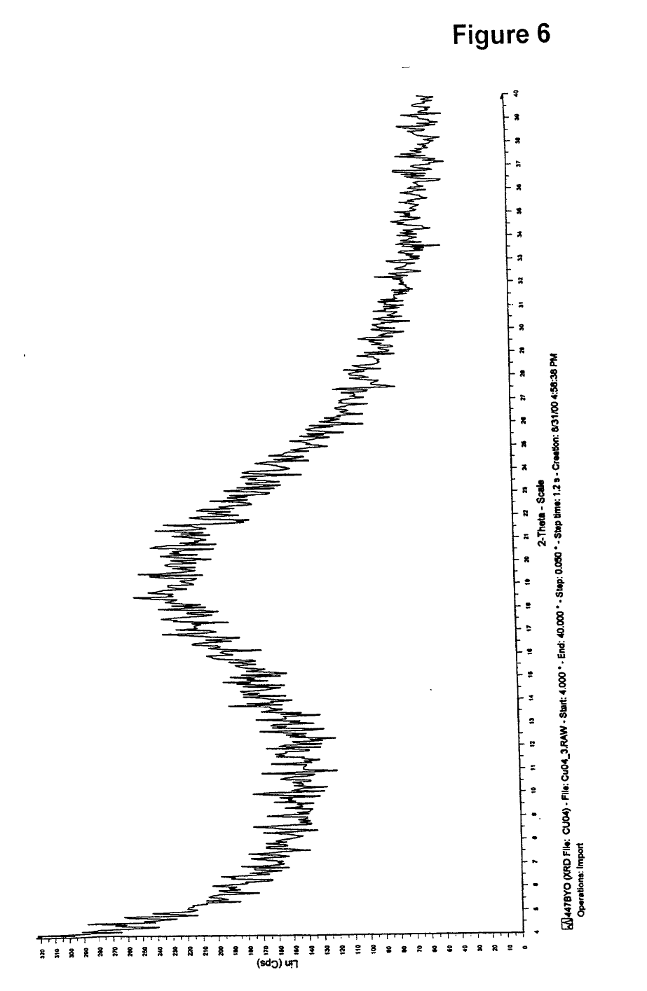

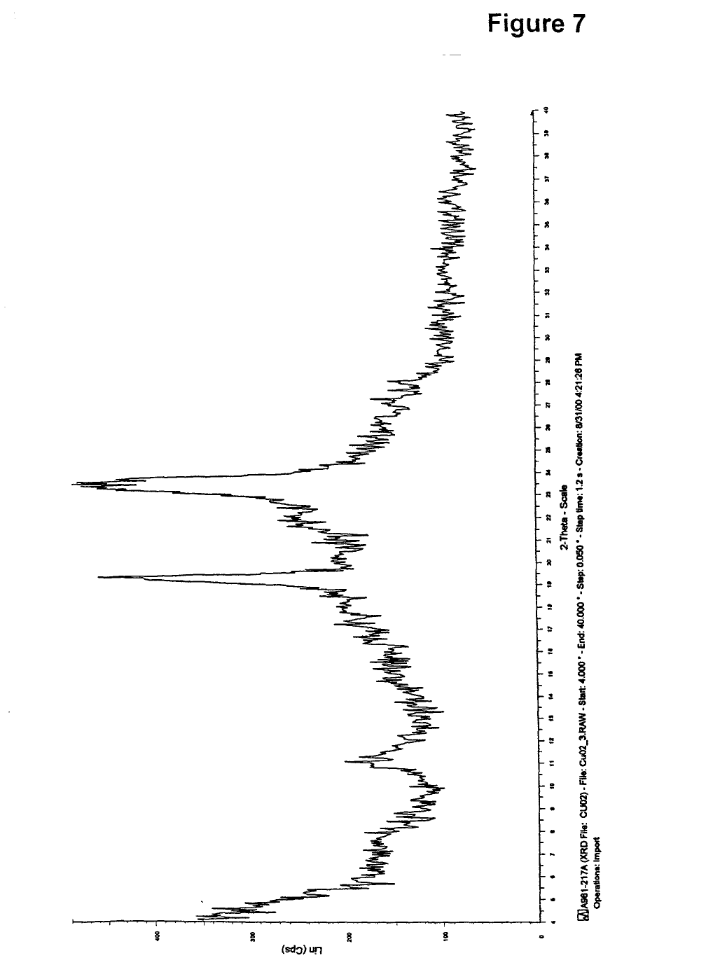

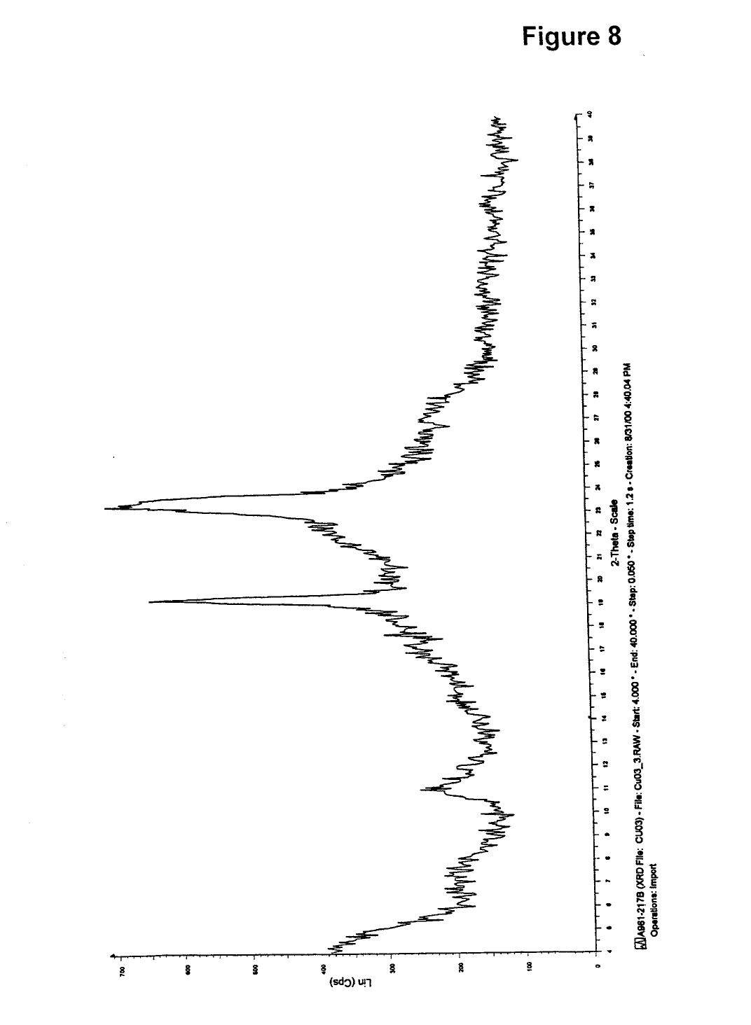

All samples were done using a Cu (λ=1.54 Å) x-ray source. The amorphous daptomycin sample did not show any peaks by x-ray powder diffraction. See FIG. 6. In contrast, the two daptomycin samples both showed peaks by x-ray powder diffraction. The diffraction angle (2θ) of the first daptomycin sample (FIG. 7) was 19.225, 23.242, 23.427 and 23.603 (degree). The diffraction angle (2θ) for the second daptomycin sample (FIG. 8) was 10.966, 19.205 and 23.344 (degree). The first crystalline daptomycin sample also showed a small peak between 10-11°. See FIG. 7.

### EXAMPLE 11

Daptomycin was dissolved in water. Sodium acetate was added to achieve a final concentration of 187 mM. Calcium chloride was added to achieve a final concentration of 28 mM. The daptomycin solution was mixed and isopropanol was added to a final concentration of 78.4%. The solution was mixed and incubated. A precipitated material was formed after incubation. The precipitated material appeared to be urchin-like crystals of approximately 60 μm diameter by optical microscopy. The material was then dried. The dry material contained approximately 30-40% salt. After drying, powder x-ray diffraction was performed. The powder x-ray diffraction did not show the presence of crystals in the dried daptomycin precipitate.

### EXAMPLE 12

One gram of daptomycin (approximately 91.5% purity as measured by HPLC) was added to 16.8 mL of distilled water and dissolved. 2.5 mL of IM calcium acetate (pH 6.1) and 60 mL of isopropanol was added. The solution was placed in a 27° C. water bath and permitted to equilibrate to temperature of the water bath. 5 mL aliquots of isopropanol were slowly added until the solution became cloudy (a total of approximately 30 mL isopropanol). The solution was incubated overnight at 27° C. to form a precipitate. The precipitate appeared to contain urchin-like crystals of approximately 60 μm by optical microscopy. See FIG. 2.

The daptomycin precipitate was poured into a pressure filter/drying funnel and filtered by gravity. The precipitate was washed twice with 25 mL each time of a washing solution (80% isopropanol and 20% solution A where solution A consists of 18mL of water and 2mL of glacial acetic acid) and allowed to drip by gravity overnight. The precipitate was then transferred to a desiccator and dried under vacuum. After drying, powder x-ray diffraction was performed. The powder x-ray diffraction did not show the presence of crystals in the dried daptomycin precipitate. However, purity analysis of the precipitated material by HPLC showed that the material was 98.2% pure daptomycin. Significantly, the daptomycin preparation after precipitation has significantly less anhydro-daptomycin than the daptomycin preparation before precipitation.

Without wishing to be bound by any theory, applicants believe that the conditions used to precipitate the daptomycin in Examples 11 and 12 actually produce a crystalline form of daptomycin but that the subsequent washing steps and/or drying steps cause the crystalline daptomycin to revert to a non-crystalline form. Nonetheless, the non-crystalline daptomycin is still crystal-like as shown in FIG. 3 by the birefringence of a crystal sample in polarized light.

### EXAMPLE 13

A fermentation culture of S. roseosporus NRRL Strain 15998 is conducted in a controlled decanoic acid feed fermentation at levels that optimize the production of the antibiotic while minimizing the production of contaminants. The residual decanoic acid feed is measured by gas chromatography and the target residual level is 10 ppm decanoic acid from the start of induction (approximately at hour 30) until harvest. Centrifugation of the culture and subsequent analysis of the clarified broth are used to measure the production of daptomycin by HPLC. The harvest titer is typically between 1.0 and 3.0 grams per liter of fermentation broth.

The fermentation culture is harvested either by microfiltration using a Pall-Sep or equivalent microfiltration system, or by full commercial-scale centrifugation and depth filter. The clarified broth is applied to an anion exchange resin, Mitsubishi FP-DA 13, washed with of 30 mM NaCl at pH 6.5 and eluted with of 300 mM NaCl at pH 6.0-6.5. Alternatively, the FP-DA 13 column is washed with of 30 mM NaCl at pH 6.5 and eluted with of 300 mM NaCl at pH 6.0-6.5. The pH is adjusted to 3.0-4.8 and the temperature is adjusted to 2-15° C.

Under these conditions, daptomycin forms a micelle. The micellar daptomycin solution is filtered-washed using a 10,000 NMW ultrafilter (AG Technology Corp. UF hollow fiber or equivalent) in any configuration. The daptomycin micelles are retained by the filter, but a large number of impurities are eliminated because they pass through the 10,000 NMW filter. Ultrafiltration of daptomycin micelles increases daptomycin purity to approximately 80-90%.

The daptomycin preparation is then crystallized or precipitated under sterile conditions using one of the methods described above. In a preferred embodiment, the daptomycin is crystallized or precipitated according to the protocol described in Examples 7, 8 or 12 except that it can be scaled up for large preparation of daptomycin. The crystalline or crystal-like daptomycin is separated from the crystallization/precipitation solution by filtration, preferably by vacuum filtration. The crystalline or crystal-like daptomycin is washed with washing solution (see Example 3). The crystalline or crystal-like daptomycin is then vacuum dried under sterile conditions using a 0.65 m³ Klein Hastelloy-B double cone vacuum dryer or equivalent apparatus.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method for preparing daptomycin, comprising the steps of providing an amorphous form of daptomycin and crystallizing the daptomycin from a crystallization solution comprising a cation from a salt, a buffer, an organic precipitant, and a low molecular weight alcohol.

2. The method according to claim 1, wherein the buffer is selected from the group consisting of HEPES, Tris HCl, imidazole, MES, CHES, a citrate salt and a cacodylate salt.

3. The method according to claim 1, wherein the alcohol is selected from the group consisting of ethylene glycol, propylene glycol, t-butanol, glycerol, isopropanol, 1,4-butanediol, 1,2-propanediol and methanol.

4. The method according to claim 1, wherein the organic precipitant is polyethylene glycol or polyethylene glycol monomethyl ether.

5. The method according to claim 1, wherein the crystallizing solution further comprises a divalent cation.

6. The method according to claim 5, wherein the divalent cation is calcium, zinc or magnesium.

7. The method according to claim 1, wherein the pH of the crystallization solution is in the range of 5 to 8.5.

8. The method according to claim 7, wherein the pH of the crystallization solution is in the range of 5.5 to 7.5.

9. The method according to claim 8, wherein the pH of the crystallization solution is in the range of 5.9 to 6.6.

10. The method according to claim 1, wherein the crystallization is done by the hanging drop method or by batch crystallization.

11. The method according to claim 1, wherein a crystal of the daptomycin is an urchin-like or a cluster of needles form.

12. The method according to claim 1, wherein a crystal of the daptomycin is a rod-like form.

13. The method according to claim 1, further comprising the step of collecting the daptomycin crystals.

14. The method according to claim 13, wherein said collecting is done by centrifugation, precipitation or filtration.

15. The method according to either of claims 1 or 14, further comprising washing the crystalline daptomycin.

16. The method according to claim 1, wherein the daptomycin is at a starting purity of at least 90%.

17. The method according to claim 1, wherein the daptomycin is at a starting purity of at least 93%.

18. The method according to claim 1, wherein said crystallizing is performed at a temperature below 20° C.

19. The method according to claim 18, wherein said crystallizing is performed at about 4° C.

20. The method according to claim 1, wherein said crystallizing is performed at above 20° C.

21. The method according to claim 1, wherein said crystallizing is performed with stirring.

22. A method for preparing a crystalline or crystal-like daptomycin, comprising the steps of
a) providing a solution comprising daptomycin, a salt comprising a monovalent or divalent cation, a pH buffering agent and a low molecular weight or polyhydric alcohol; and
b) allowing the daptomycin to crystallize or precipitate from the solution to obtain a crystalline or crystal-like daptomycin preparation, respectively.

23. The method according to claim 22, wherein the buffering agent is selected from the group consisting of HEPES, Tris HCl, imidazole, MES, CHES, sodium acetate, calcium acetate, a citrate salt and a cacodylate salt.

24. The method according to claim 22, wherein the alcohol is selected from the group consisting of ethylene glycol, propylene glycol, t-butanol, glycerol, isopropanol, 1,4-butanediol, 1,2-propanediol and methanol.

25. The method according to claim 24, wherein the alcohol is isopropanol.

26. The method according to claim 22, wherein the salt comprises a divalent cation.

27. The method according to claim 26, wherein the divalent cation is a magnesium cation, a zinc cation or a calcium cation.

28. The method according to claim 27, wherein the divalent cation is a calcium cation.

29. A method for preparing a crystalline or crystal-like daptomycin, comprising the steps of
a) providing a solution comprising daptomycin, a pH buffering agent that is a salt comprising a monovalent or divalent cation, and a low molecular weight or polyhydric alcohol; and
b) allowing the daptomycin to crystallize or precipitate from the solution to obtain a crystalline or crystal-like daptomycin preparation, respectively.

30. The method according to claim 29, wherein the buffering agent comprises a divalent cation selected from a calcium cation or a magnesium cation.

31. The method according to claim 22 or claim 29, wherein the pH of the solution is in the range of 5.0 to 9.5.

32. The method according to claim 31, wherein the pH of the solution is in the range of 5.5 to 7.5.

33. The method according to claim 32, wherein the pH of the solution is in the range of 5.9 to 6.3.

34. The method according to either of claims 22 or 29, wherein said crystallizing or precipitating step is done at a temperature of 0-30° C.

35. The method according to claim 34, wherein the temperature is 23-28° C.

36. The method according to claim 29, wherein the solution comprises calcium acetate pH 6.1 and isopropanol.

37. The method according to claim 36, wherein said crystallizing or precipitating step comprises adding isopropanol until the mixture becomes cloudy.

38. The method according to claim 37, wherein said crystallizing or precipitating step is done for a period of time of from one hour to three weeks.

39. The method according to claim 22, wherein said crystallizing or precipitating is done by batch crystallization or batch precipitation, respectively.

40. The method according to claim 22 or claim 29, further comprising the step of collecting the crystalline or crystal-like daptomycin.

41. The method according to claim 40, wherein said collecting step is performed by filtration or centrifugation.

42. The method according to claim 41, wherein said collecting is performed by filtration.

43. The method according to claim 40, further comprising the step of washing the crystalline or crystal-like daptomycin.

44. The method according to claim 22 or claim 29, wherein the crystalline or crystal-like daptomycin has an urchin-like form.

45. The method according to claim 22 or 29, wherein the daptomycin has a purity before crystallizing or precipitating of no greater than 90% and has a purity after crystallization or precipitation of at least 95%.

46. The method according to claim 45, wherein the daptomycin has a purity before crystallizing or precipitating of no greater than 80% and has a purity after crystallization or precipitation of at least 95%.

47. The method according to claim 45, wherein the daptomycin has a purity before crystallizing or precipitating of no greater than 60% and has a purity after crystallization or precipitation of at least 95%.

48. The method according to claim 45, wherein the daptomycin has a purity before crystallizing or precipitating of no greater than 40% and has a purity after crystallization or precipitation of at least 95%.

49. The method according to claim 45, wherein the daptomycin is at a starting purity of no greater than 10% and has a purity after crystallization or precipitation of at least 95%.

50. The method according to any one of claims 46-50, wherein the daptomycin has a purity after crystallization or precipitation of at least 96%.

51. The method according to any one of claims 46-50, wherein the daptomycin has a purity after crystallization or precipitation of at least 97%.

52. The method according to any one of claims 46-50, wherein the daptomycin has a purity after crystallization or precipitation of at least 98%.

53. A method for preparing a purified daptomycin, comprising the steps of
a) providing a solution comprising daptomycin, a pH buffering agent that is a salt comprising a monovalent or divalent cation, and a low molecular weight or polyhydric alcohol; and
b) allowing the daptomycin to crystallize or precipitate from the solution to obtain a purified daptomycin preparation.

54. The method according to claim 53, wherein the purified daptomycin preparation is at least 95% pure.

55. The method according to claim 54, wherein said purified daptomycin preparation is at least 96% pure.

56. The method according to claim 55, wherein said purified daptomycin preparation is at least 97% pure.

57. The method according to claim 56, wherein said purified daptomycin preparation is at least 98% pure.
